(19) United States
(12) Patent Application Publication
(10) Pub. No.: US 2008/0319202 A1
(43) Pub. Date: Dec. 25, 2008

(54) FLUID STORAGE AND DISPENSING METHODS AND APPARATUS

(76) Inventors: Douglas L. Gin, Longmont, CO (US); Jason E. Bara, Boulder, CO (US); Richard D. Noble, Boulder, CO (US)

Correspondence Address: GREENLEE WINNER AND SULLIVAN P C, 4875 PEARL EAST CIRCLE, SUITE 200, BOULDER, CO, 80301, US

(21) Appl. No.: 12/041,574

(22) Filed: Mar. 3, 2008

Related U.S. Application Data
(63) Continuation of application No. 11/772,174, filed on Jun. 30, 2007.
Application No. 11/772,174 is a continuation of application No. PCT/US07/72615, filed on Jun. 30, 2007.
(60) Provisional application No. 60/806,524, filed on Jul. 3, 2006.
(60) Provisional application No. 60/892,807, filed on Mar. 2, 2007.
(60) Provisional application No. 60/806,524, filed on Jul. 3, 2006.
(60) Provisional application No. 60/892,807, filed on Mar. 2, 2007.
(60) Provisional application No. 60/892,807, filed on Mar. 2, 2007.

(51) Int. Cl. C07D 233/54; C07D 403/02

(52) U.S. Cl. 548/300.1; 548/313.7

(57) ABSTRACT

A method and device for storing and dispensing a fluid includes providing a vessel configured for selective dispensing of the fluid therefrom. Provided within a vessel is a nanocomposite material comprising an imidazolium surfactant and an integral solvent that is essential to the formation of the nanocomposite material. The fluid is contacted with the nanocomposite material for take-up of the fluid by the polymerized nanocomposite material. The fluid is released from the nanocomposite material and dispensed from the vessel.

### CROSS REFERENCE TO OTHER APPLICATIONS

This application claims benefit of priority to two provisional U.S. Application Nos. 60/806,524, filed Jul. 3, 2006, and 60/892,807, filed Mar. 2, 2007, the disclosures of which are fully incorporated herein by reference.

### BACKGROUND OF THE INVENTION

1. Field of the Invention

The present invention relates to a method of storing a fluid, and more particularly to a vessel having a nanocomposite material, that may optionally be polymerized, comprising a surfactant and an integral solvent that is essential to the formation of the nanocomposite material. The surfactant may be, but is not limited to, a polymerizable cationic imidazolium surfactant that can form ordered, nanostructured, phase-segregated lyotropic liquid crystal (LLC) phases when mixed with either water, room temperature ionic liquids (RTILS), other solvents or mixtures of said liquids. The LLC phases formed may be, but are not limited to, special bicontinuous cubic (Q) type phases. LLC phases with other geometries are also applicable.

2. Description of the State of the Art

Many industrial processes require a reliable source of process gases for a wide variety of applications. Often these gases are stored in cylinders or vessels and then delivered to the process under controlled conditions from the cylinder. For example, the silicon semiconductor manufacturing industry, as well as the compound semiconductor industry, uses a number of hazardous specialty gases such as diborane, stibene, phosphine, arsine, boron trifluoride, hydrogen chloride, and tetrafluoromethane for doping, etching, thin-film deposition, and cleaning. These gases pose significant safety and environmental challenges due to their high toxicity and reactivity. Additionally, storage of hazardous gases under high pressure in metal cylinders is often unacceptable because of the possibility of developing a leak or catastrophic rupture of the cylinder, cylinder valve, or downstream component.

In order to mitigate some of these safety issues associated with high pressure cylinders, there is a need for a low pressure storage and delivery system. Additionally, some gases, such as diborane, tend to decompose when stored for a period of time. Thus, it would be useful to have a way to store unstable gases in a manner that reduces or eliminates the decomposition.

It is also desirable to have a method of removing impurities from gases, particularly in the semiconductor industry. The growth of high quality thin film electronic and optoelectronic cells by chemical vapor deposition or other vapor-based techniques is inhibited by a variety of low-level process impurities which are present in gas streams involved in semiconductor manufacturing or are contributed from various components such as piping, valves, mass flow controllers, filters, and similar components. These impurities can cause defects that reduce yields by increasing the number of rejects, which can be very expensive.

Chemical impurities may originate in the production of the source gas itself, as well as in its subsequent packaging, shipment, storage, handling, and gas distribution system. Although source gas manufacturers typically provide analyses of source gas materials delivered to the semiconductor manufacturing facility, the purity of the gases may change because of leakage into or outgassing of the containers, e.g. gas cylinders, in which the gases are packaged. Impurity contamination may also result from improper gas cylinder changes, leaks into downstream processing equipment, or outgassing of such downstream equipment. Source gases may include impurities, or impurities may occur as a result of decomposition of the stored gases. Impurities can also occur as a result of chemical reaction between the container surface and the fluid. Furthermore, the impurity levels within the gas container may increase with length of storage time and can also change as the container is consumed by the end user.

Thus, there remains a need for a low pressure storage and delivery device that is also able to remove contaminants from gases, particularly to very low levels.

### SUMMARY OF THE INVENTION

In one aspect of the invention, a method of storing and dispensing a fluid is provided. The method includes providing a vessel having a nanocomposite material within, that may be optionally polymerized, wherein the vessel is configured for maximized storage of the fluid therein. The nanocomposite material is configured to maximize its surface area and comprises a surfactant, such as but not limited to, a polymerizable cationic imidazolium and an integral solvent that is essential to the formation of the polymerized nanocomposite material. The solvent may be, but is not limited to, either water, room temperature ionic liquids (RTILS), other solvents or mixtures thereof and when mixed with a cationic imidazolium surfactant, nanostructured, phase-separated lyotropic liquid crystal (LLC) phases are formed. Of particular interest are bicontinuous cubic (Q) LLC phases which possess high accessible surface area due to 3-D interconnected solvent and LLC surfactant domains. However, other nanostructured LLC phases such as the inverted hexagonal, lamellar, and other types of cubic LLC phases formed by the aforementioned polymerizable cationic imidazolium surfactants, are also of interest. The resulting polymerized nanocomposite material is positioned within the vessel and the fluid is contacted with the polymerized nanocomposite material for take-up of the fluid by the polymerized nanocomposite material. The fluid is later released from the polymerized nanocomposite material and dispensed from the vessel. The fluid may be selected from alcohols, aldehydes, amines, ammonia, aromatic hydrocarbons, arsenic pentafluoride, arsine, boron trichloride, boron trifluoride, carbon disulfide, carbon monoxide, carbon sulfide, diborane, dichlorosilane, digermane, dimethyl disulfide, dimethyl sulfide, disilane, ethers, ethylene oxide, fluorine, germane, germanium methoxide, germanium tetrafluoride, hafnium methylethylamide, hafnium t-butoxide, halogenated hydrocarbons, halogens, hexane, hydrogen, hydrogen cyanide, hydrogen halogenides, hydrogen selenide, hydrogen sulfide, ketones, mercaptans, nitric oxides, nitrogen, nitrogen trifluoride, organometallics, oxygenated-halogenated hydrocarbons, phosgene, phosphorus trifluoride, n-silane, pentakisdimethylamino tantalum, silicon tetrachloride, silicon tetrafluoride, stibine, styrene, sulfur dioxide, sulfur hexafluoride, sulfur tetrafluoride, tetramethyl cyclotetrsiloxane, titanium diethylamide, titanium dimethylamide, trichlorosilane, trimethyl silane, tungsten hexafluoride, and mixtures thereof.

The surfactants are gemini (i.e., two headed), cationic imidazolium surfactants (nonpolymerizable and polymerizable versions) based on RTIL compounds, that can form bicontinuous cubic LLC phases when mixed with RTILs, water or mixtures thereof as the solvent. The surfactant has the general formulation:

HₙXₙL₍ₙ₋₁₎Yₙ Formula 1

where n is greater than or equal to 2; H is a hydrophilic head group comprising a five membered aromatic ring containing two nitrogens (e.g. an imidazolium ring); X is an anion, L is a spacer or linking group which connects the rings, and Y is a hydrophobic tail group attached to each ring and having at least 10 carbon atoms which optionally comprise a polymerizable group P. Each spacer L is attached to a first nitrogen atom in each of the two linked rings. The attachment may be through a covalent or a non-covalent bond such as an ionic linkage. Each hydrophobic tail group Y is attached to the second (other) nitrogen atom in each ring. The combination of the hydrophilic head group H, the linker L, and the hydrophobic tail Y form an imidazolium cation. Hydrophobic tails may also be attached to one or more carbon atoms of the ring.

The anion, X, is a standard anion used in preparing room temperature ionic liquids. These anions include, but are not limited to Br⁻, BF₄⁻, Cl⁻, I⁻, CF₃SO₃⁻, Tf₂N⁻, (any other large fluorinated anions), PF₆⁻, DCA⁻, MeSO₃⁻, and TsO⁻. In an embodiment, the anion X is selected from the group consisting of Br⁻, and BF₄⁻.

The spacer L can be an alkyl group, an ether group, an amide, an ester, an anhydride, a phenyl group, a perfluoroalkyl, a perfluoroether, or a siloxane. In an embodiment, L is an alkyl group having from 1 to about 12 carbons, or an ether group having from about 1 to about 6 ethers. In an embodiment, L is an ether group having from 1 to 3 ethers. In addition, the spacer L can include a pendant functional group such as a catalytic group or a molecule receptor.

Y is a hydrophobic tail group having at least 10 carbon atoms. The tail group may be linear or branched. A linking group may be placed between the tail and the ring. In an embodiment, Y is a linear alkyl chain. In another embodiment, Y comprises a polymerizable group. Suitable polymerizable groups include acrylate, methacrylate, diene, vinyl, (halovinyl), styrenes, vinylether, hydroxyl groups, epoxy or other oxiranes (halooxirane), dienoyls, diacetylenes, styrenes, terminal olefins, isocyanides, acrylamides, and cinamoyl groups. In an embodiment, the polymerizable group is an acrylate, methacrylate or diene group.

In another embodiment, n=2 and the surfactant composition has the general formula:

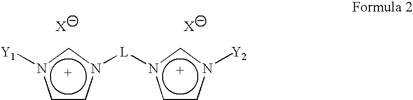

In another embodiment, n=2 and the surfactant composition has the general formula:

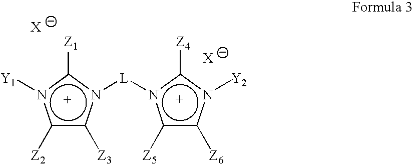

In Formula 3, Z₁ through Z₆ are individually selected from the group consisting of hydrogen and hydrophobic tail groups having at least 10 carbon atoms which optionally comprise a polymerizable group P.

Variance in the chemical character of the hydrophobic tail attached to the nitrogen can be used to tune LLC phase structure and curvature as well as surface properties. Attachment of a hydrophobic tail to one or more carbon atoms in the ring can be of further utility in tuning the structure-property relationships. The nature and concentration of these tails may affect the surface, the structure, or other aspects of the LLC phase even to the point of altering its symmetry. Thus any geometries or symmetries listed herein are representative, and not intended as an exhaustive delineation of potential structures that may limit the scope of the invention.

The surfactant compositions may also be described as shown in FIG. 1. In an embodiment t is between 1 and 12 or u is between 1 and 6. In another embodiment, surfactants which form the bicontinuous cubic phase have particular linking groups, such as pendant groups R, wherein R=(CH₂)ₜ, t=6, and X⁻=BF₄⁻. Surfactants which form the bicontinuous cubic phase also can have R=(OCH₂)ᵤ and u=1 or 2.

The solvent selected is thus dependent upon the surfactant used and may be selected from either water, room temperature ionic liquids or mixtures thereof. For example, [emim][BF₄] is a good match for the liquid crystals that have 2 BF₄⁻ anions associated with them. As is know in the art, emim stands for ethyl methyl imidazolium. In an embodiment, the concentration of the surfactant or monomer is between 10% and 100%.

The foregoing paragraphs have been provided by way of general introduction, and are not intended to limit the scope of the following claims. The presently preferred embodiments, together with further advantages, will be best understood by reference to the following detailed description taken in conjunction with the accompanying drawings.

### BRIEF DESCRIPTION OF THE DRAWINGS

The accompanying drawings, which are incorporated in and form a part of the specifications, illustrate the preferred embodiments of the present invention, and together with the description serve to explain the principles of the invention.

FIG. 1 schematically depicts the imidazolium-based gemini surfactants and polymerizable surfactants that form Q LLC phases with RTILs and water as the polar solvent.

FIG. 2 shows an embodiment of a vessel for storing a fluid in a polymerized nanocomposite material.

FIG. 3 shows another embodiment of a device for storing a fluid in a polymerized nanocomposite material.

FIG. 4 shows an embodiment of a device for storing a fluid with a polymerized nanocomposite material.

FIG. 5 shows another embodiment of a device for storing a fluid with a polymerized nanocomposite material.

### DETAILED DESCRIPTION OF THE PRESENT INVENTION

The present invention is directed to the use of nanocomposite materials to store a fluid material such as a gas or liquid. The nanocomposite material may be polymerized and will be throughout this disclosure referenced as a polymerized nanocomposite material. The polymerized nanocomposite material is configured to maximize its surface area and comprises a surfactant, such as but not limited to, a polymerizable cationic imidazolium and an integral solvent that is essential to the formation of the polymerized nanocomposite material. A vessel is configured for the selective dispensing of the fluid and contains a polymerized nanocomposite material. The fluid is contacted with the polymerized nanocomposite material for take-up of the fluid by the polymerized nanocomposite material. This allows storage of the fluid for a period of time. In one embodiment, the material in the storage vessel is at high pressure, for example up to about 4000 psi, preferably up to at least about 2000 psi. In another embodiment, the pressure of the material in the storage vessel is at around atmospheric pressure, which allows for safer storage conditions compared to high-pressure storage vessels.

The polymerized nanocomposite material may also be used to store unstable fluids such as diborane which tend to decompose. The storage in the polymerized nanocomposite material can reduce or eliminate the decomposition of the unstable fluids.

In an embodiment, a polymerized nanocomposite material for use in the methodology of present invention is formed by mixing a solvent with a surfactant composition having the general formulation:

HₙXₙL₍ₙ₋₁₎Yₙ Formula 1

Where n is greater than or equal to 2; H is a hydrophilic head group comprising a five membered aromatic ring containing two nitrogens (e.g. an imidazolium ring); X is an anion, L is a spacer or linking group which connects the rings, and Y is a hydrophobic tail group attached to each ring and having at least 10 carbon atoms which optionally comprise a polymerizable group P. Each spacer L is attached to a first nitrogen atom in each of the two linked rings. The attachment may be through a covalent or a non-covalent bond such as an ionic linkage. Each hydrorophobic tail group Y is attached to the second (other) nitrogen atom in each ring. The combination of the hydrophilic head group H, the linker L, and the hydrophobic tail Y form an imidazolium cation. Hydrophobic tails may also be attached to one or more carbon atoms of the ring.

The anion, X, is a standard anion used in preparing room temperature ionic liquids. These anions include, but are not limited to Br⁻, BF₄⁻, Cl⁻, I⁻, CF₃SO₃⁻, Tf₂N⁻, (any other large fluorinated anions), PF₆⁻, DCA⁻, aryl or alkyl sulfonates, such as MeSO₃⁻, and TsO⁻. In an embodiment, the anion X is selected from the group consisting of Br⁻, and BF₄⁻.

The spacer L can be an alkyl group, an ether group, an amide, an ester, an anhydride, a phenyl group, a perfluoroalkyl, a perfluoroether, or a siloxane. In an embodiment, L is an alkyl group having from 1 to about 12 carbons, or an ether group having from about 1 to about 6 ethers. In an embodiment, L is an ether group having from 1 to 3 ethers. In addition, the spacer L can include a pendant functional group such as a catalytic group or a molecule receptor.

Y is a hydrophobic tail group having at least 10 carbon atoms. The tail group may be linear or branched. A linking group may be placed between the tail and the ring. In an embodiment, Y is a linear alkyl chain. In another embodiment, Y comprises a polymerizable group. Suitable polymerizable groups include acrylate, methacrylate, diene, vinyl, (halovinyl), styrenes, vinylether, hydroxyl groups, epoxy or other oxiranes (halooxirane), dienoyls, diacetylenes, styrenes, terminal olefins, isocyanides, acrylamides, and cinamoyl groups. In an embodiment, the polymerizable group is an acrylate, methacrylate or diene group.

In an embodiment, n=2 and the surfactant composition has the general formula:

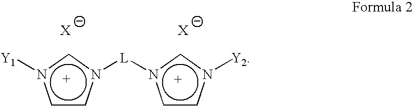

Specific examples of Formula 2 include, but are not limited to, materials having two imidazolium cations tethered to each other. Such materials shall be herein referred to as “gemini” systems. Each cation is functionalized with a single polymerizable group, resulting in a system that is self-crosslinking upon polymerization. Examples are shown below.

Formula 2.1 is a general depiction of a gemini imidazolium system, with styrene as a polymerizable group. The linkage between each imidazolium ring and its respective styrene group is at least one carbon (j≧1).

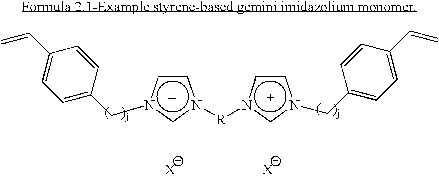

Formula 2.2 is a general depiction of an imidazolium monomer, with an acrylate as a polymerizable group. The linkage between the imidazolium ring and the ester is at least two carbons (n≧2).

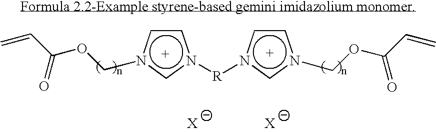

Other polymerizable groups are possible, but these two are most preferable, as they can be easily added and polymerized controllably. Formulas 2.3a and 2.3b show possible formulations for the tether group (R) on either type of system.

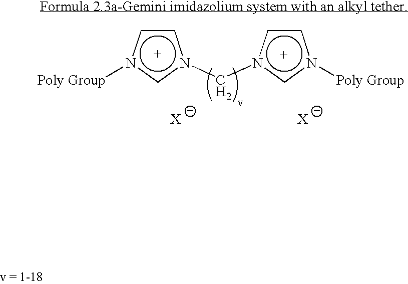

In formula 2.3a the tether group (R) is an alkyl chain with a formula range of CH₂—C₁₈H₃₆.

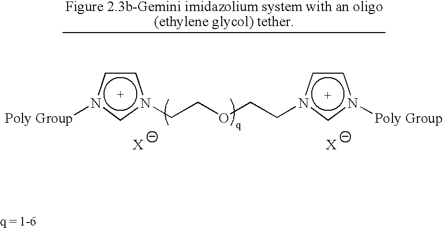

In formula 2.3b the tether group (R) is an oligo (ethylene glycol) chain with a formula range of C₄H₈—C₁₄H₂₈O₆. Other possibilities for the tether group (R) include, but are not limited to linkages containing perfluoroalkyl, siloxane, nitrile, ester, aromatic and cyclic units.

Both anions (X) are typically chosen from (but not necessarily limited to) the groups shown below.

The polymerization of these monomers may be initiated either through a photo or thermal pathway. Additional crosslinker molecules (e.g. divinylbenzene, 1,6-hexandioldiacrylate, etc.) may be added prior to initiation for copolymerization to influence mechanical properties of the resulting polymers.

A miscible, non-polymerizable room temperature ionic liquid (RTIL) may be blended with above materials to form a composite. Said addition of RTIL may occur before or after the polymerization reaction is carried out, to control properties such as glass transition temperature (T_{g}) and to influence the solubility and diffusion of various solutes (i.e. gases and vapors) in polymers produced from these monomers.

In another embodiment, n=2 and the surfactant composition has the general formula:

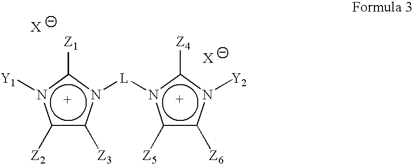

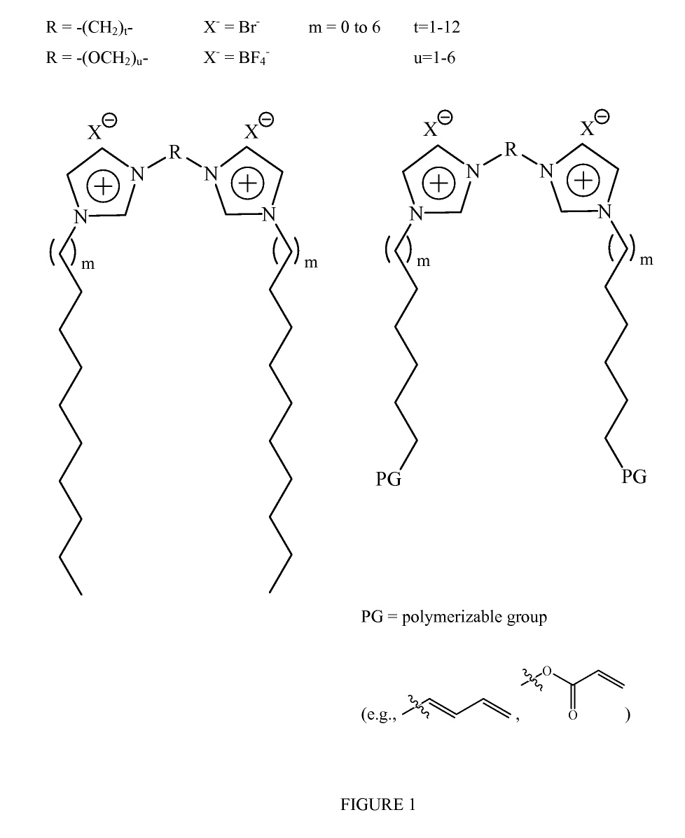

In Formula 3, Z₁ through Z₆ are individually selected from the group consisting of hydrogen and hydrophobic tail groups having at least 10 carbon atoms which optionally comprise a polymerizable group P. Attachment of a hydrophobic tail to one or more carbon atoms in the ring in addition to the hydrophobic tail attached to the nitrogen can be of further utility in tuning the structure, curvature, symmetry or geometry of the LLC phase, as well as binding energy, capacity, uptake and release kinetics or other surface properties. The surfactant compositions may also be described as shown in FIG. 1. In an embodiment t is between 1 and 12 or u is between 1 and 6. In another embodiment, surfactants which form the bicontinuous cubic phase have R=(CH₂)ₜ, t=6, and X⁻=BF₄⁻. Surfactants which form the bicontinuous cubic phase also can have R=(OCH₂)ᵤ and u=1 or 2.

In another embodiment monomers for forming linear polymers may be utilized and these materials would have an imidazolium cation, functionalized with a single polymerizable group as shown below:

Formula 4 is a general depiction of an imidazolium monomer, with styrene as a polymerizable group. The linkage between the two phenyl group and imidazolium ring is at least one carbon (j≧1).

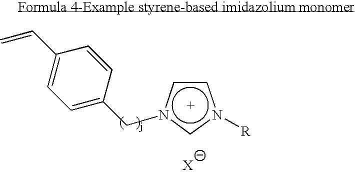

Formula 4.1 is a general depiction of an imidazolium monomer, with an acrylate as a polymerizable group. The linkage between the imidazolium ring and the ester is at least two carbons (n≧2).

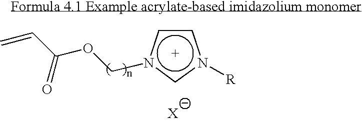

While other polymerizable groups are possible, these two are most preferable, as they can be easily added and polymerized controllably.

Formulas 4.2a and 4.3b show possible formulations for the non-polymerizable, pendant group (R) on either type of system.

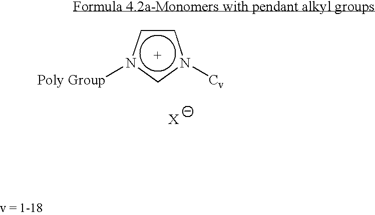

In formula 4.2a the pendant group (R) is an alkyl chain with a formula range of CH₃—C₁₈H₃₇.

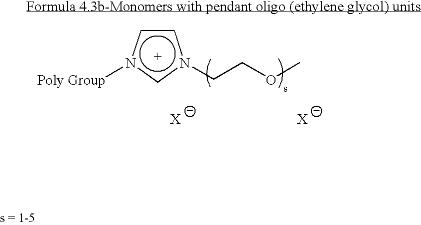

In formula 4.3b the pendant group (R) is an oligo (ethylene glycol) unit with a formula range of C₃H₇O—C₁₁H₂₃O₅. Other possibilities for the pendant group (R) include, but are not limited to, perfluoroalkyl, siloxane, nitrile, ester, aromatic and cyclic units.

The anion (X) is typically chosen from (but not necessarily limited to) the following groups:

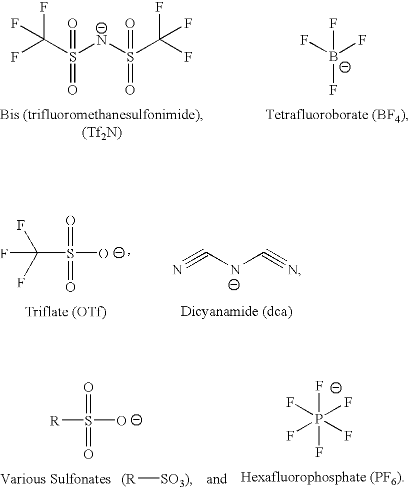

The polymerization of these monomers may be initiated either through a photo or thermal pathway. Additional crosslinker molecules (e.g. divinylbenzene, 1,6-hexandioldiacrylate, etc.) may be added prior to initiation for copolymerization to influence mechanical properties of the resulting polymers.

A miscible, non-polymerizable room temperature ionic liquid (RTIL) may be blended with above materials to form a composite. Said addition of RTIL may occur before or after the polymerization reaction is carried out, to control properties such as glass transition temperature (T_{g}) and to influence the solubility and diffusion of various solutes (i.e. gases and vapors) in polymers produced from these monomers.

The LLC phase in the polymerized nanocomposite material for use in the present invention may be formed by polymerization of the polymerizable LLC monomer tails. Polymerization is performed by chemical reaction, such as a free radical polymerization reaction. Alternatively polymerization may be initiated by irradiation with light of appropriate wave length (i.e., photoinitiated), by introduction of a chemical reagent or catalyst and/or by thermal initiation. Formation of these polymerized nanocomposite materials is disclosed in U.S. Application No. 60/806,524, which is incorporated herein in its entirety.

Ionic liquids are a relatively new class of materials which can offer such physical properties as extremely low vapor pressure, high thermal stability, and low viscosity. Generally, ionic liquids consist of a bulky, asymmetric cation and an inorganic anion. The bulky, asymmetric nature of the cation prevents tight packing, which decreases the melting point. Due to the wide variety of cations and anions possible for such ion pairs, a wide range of gas solubilities is conceivable, for a variety of inorganic and organic materials. The physical properties of ionic liquids can include good dissolution properties for most organic and inorganic compounds; high thermal stability, non-flammability; negligible vapor pressure; low viscosity, compared to other ionic materials; and recyclability.

The wide range of chemical functionalities available with ionic liquids offers possibilities for gas delivery and control. For example, ionic liquids may provide the capability to control the release of a gas and/or its impurities via solubility control with temperature or pressure. This may enable the storage of a gas and its impurities, while selectively releasing only the desired gas by changing certain parameters, such as temperature or pressure, leaving the impurities behind. Thus there is potential for an ionic liquid system that could function as a 2-in-1 system, providing both storage and purification in one container.

Ionic liquids can have a stabilizing effect on intermediate reaction species in organic synthesis and catalysis. Thus, ionic liquids can offer stabilizing effects for unstable gas molecules. Thus, utilization with even a small amount of ionic liquid, can reduce or eliminate the decomposition of the unstable fluids. Storage of a gas or other fluid in an ionic liquid may also be combined with the previously mentioned purification system to provide a 3-in-1 storage, stabilization, and purification system.

The affinity of a gas in an ionic liquid varies with physical parameters such as temperature and pressure. However, it is also evident that the gas affinity obtained depends on the ionic liquid used, particularly the anion and cation used. While not intending to be bound by any particular theory, the current understanding is that the anion has a strong influence on gas solubility. Specifically, the greater the interaction between the anion and fluid, the greater the uptake of the fluid dissolution appears to occur. The cation seems to be of secondary influence. Thus, several properties of the anion, the cation, and the stored fluid play a role in these interactions. In addition, mixtures of different ionic liquids could result in unexpected high capacities of various fluids.

The purity of an ionic liquid is also believed to have an impact on its behavior. Ionic liquids which have been dried or baked, thus leaving them substantially anhydrous, may exhibit greater increased capacity for taking up fluid components. In addition, the presence of water or other impurities may decrease the solubility of certain fluid components, especially those gas components that are hydrophobic.

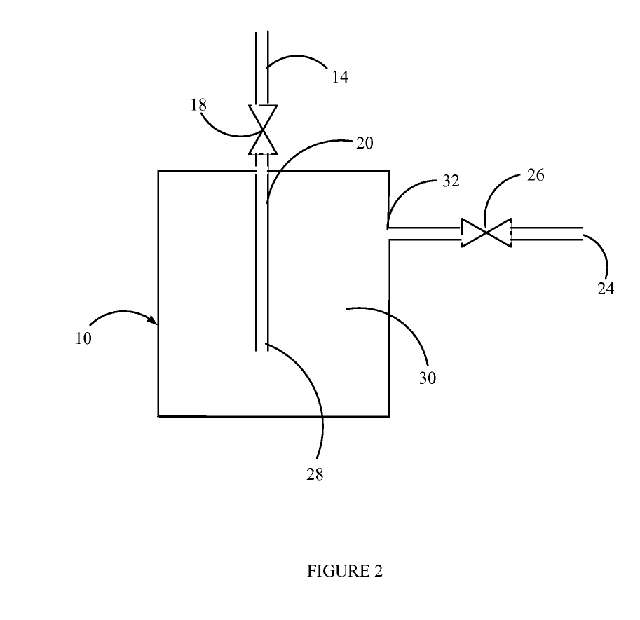

The method of storing and dispensing a fluid includes providing a vessel. One embodiment of a vessel 10 is shown in FIG. 2. Vessel 10 includes a fluid inlet 20, a polymerized nanocomposite material 30, and a fluid outlet 32. The fluid inlet 20 is connected to a fluid source 14 which is controlled by a valve 18. The polymerized nanocomposite material 30 is placed within vessel 10 prior to being welded shut. The fluid outlet 32 is controlled by valve 26. The vessel is configured for selective dispensing of the fluid therefrom. The vessel is charged with a polymerized nanocomposite material 30. A vacuum bake procedure may be conducted on vessel 10 to remove contaminants or other impurities from the polymerized nanocomposite material 30, preferably by pulling a vacuum while heating. This is done in order to remove any trace moisture and/or other volatile impurities from the polymerized nanocomposite material 30 and the fluid distribution components. The polymerized nanocomposite material 30 is allowed to cool to the desired operating temperature.

The fluid may be introduced at any suitable pressure. In one embodiment, the fluid is a gas at a temperature of about 5 psi. In another embodiment, the gas is introduced at a pressure of at least about 100 psi, preferably up to about 2000 psi or up to about 4000 psi. In one embodiment, the gas is introduced until the inlet and outlet concentrations are equivalent, indicating the polymerized nanocomposite material 30 is saturated and cannot accept any further gas under the existing conditions. At this time, the source gas flow is stopped.

In one embodiment, contacting the fluid with the polymerized nanocomposite material 30 comprises flowing the fluid mixture through the polymerized nanocomposite material 30, as shown in FIG. 2. Vessel 10 is charged with a fluid through inlet 28 and through fluid inlet 20, such as a dip tube, from whence it flows through polymerized nanocomposite material 30.

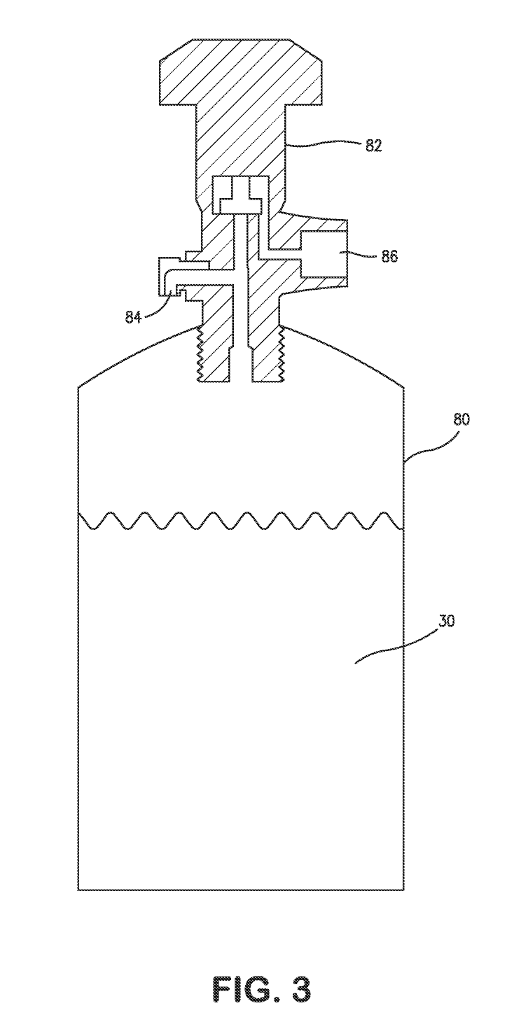

In another embodiment, the fluid is first introduced and then the vessel is mechanically agitated in order to contact the fluid with the polymerized nanocomposite material 30. FIG. 3 shows an embodiment of vessel 80 for storing a fluid in a polymerized nanocomposite material 30. The polymerized nanocomposite material 30 is put into the vessel before valve assembly 82 is inserted onto vessel 80. The fluid is then added to vessel 80 containing the polymerized nanocomposite material 30 in the conventional fashion through inlet port 84 in valve assembly 82. The vessel 80 would then be mechanically agitated to contact the fluid with the polymerized nanocomposite material 30. The fluid may be removed through outlet port 86.

In one embodiment, the fluid is a liquid. Vessel 80 shown in FIG. 3 may also be used to store a liquid in the polymerized nanocomposite material 30. The polymerized nanocomposite material 30 is put into the vessel before valve assembly 82 is inserted into vessel 80. The liquid is then added to vessel 80 in the conventional fashion through inlet port 84 in valve assembly 82. The vessel 80 would then be mechanically agitated to contact the liquid with the polymerized nanocomposite material 30. The liquid may be removed through outlet port 86.

The fluid stored within the polymerized nanocomposite material may be removed from the polymerized nanocomposite material 30 by any suitable method. The fluid is released from the polymerized nanocomposite material 30 in a substantially unreacted state. Pressure-mediated and thermally-mediated methods and sparging, alone or in combination, are preferred. In pressure-mediated evolution, a pressure gradient is established to cause the gas to evolve from the polymerized nanocomposite material 30. In one embodiment, the pressure gradient is in the range of about atmospheric pressure to about 4000 psig. In a more preferred embodiment, the pressure gradient is typically in the range from 10⁻⁷ to 600 Torr at 25° C. For example, the pressure gradient may be established between the polymerized nanocomposite material 30 in the vessel, and the exterior environment of the vessel, causing the fluid to flow from the vessel to the exterior environment. The pressure conditions may involve the imposition on the polymerized nanocomposite material 30 of vacuwn or suction conditions which effect extraction of the gas from the vessel.

In thermally-mediated evolution, the polymerized nanocomposite material 30 is heated to cause the evolution of the gas from the ionic liquid so that the gas can be withdrawn or discharged from the vessel. Typically, the temperature of the ionic liquid for thermal-mediated evolution ranges from −50° C. to 200° C., more preferably from 30° C. to 150° C. In one embodiment, the vessel containing the fluid and the polymerized nanocomposite material 30 is transported warm (i.e., around room temperature), then cooled when it is stored or used at the end user's site. In this manner, the fluid vapor pressure can be reduced at the end user's site and therefore reduce the risk of release of the gas from the vessel. Once the vessel is secured in a suitable location, the vessel can be chilled and the temperature can be controlled in such a manner as to limit the amount of gas pressure that is present in the container and piping. As the contents of the cylinder or other gas storage device are consumed, the temperature of the cylinder can be elevated to liberate the gas from the polymerized nanocomposite material 30 and to maintain the necessary amount of gas levels in the cylinder and piping.

The vessel may also be purged with a secondary gas, in order to deliver the stored primary gas. In purging, a secondary gas is introduced into the vessel in order to force the primary gas out of the polymerized nanocomposite material 30 and out of the storage container. Purging of a container can take place wherein the secondary gas is selected from a group of gases that has relatively low affinity for the ionic liquid, molecular solvent or nanocomposite solid. The secondary gas is introduced into the polymerized nanocomposite material 30 in a manner wherein the secondary gas flows through the polymerized nanocomposite material 30 and displaces the primary gas from the polymerized nanocomposite material 30. The resultant gas mixture of primary gas and secondary gas then exit the gas storage container and are delivered to a downstream component in the gas distribution system. The purging parameters should be selected such that the maximum amount of primary gas is removed from the polymerized nanocomposite material 30. This includes selection of the appropriate geometry of the vessel such that the secondary gas has an enhanced pathway for the interaction or contact between the secondary fluid and the polymerized nanocomposite material 30. In practice, this could be use of a long and narrow storage container wherein the secondary fluid is introduced at the bottom of the container and the outlet of the container is near the top.

In an additional embodiment wherein the nanocomposite material contains a liquid, a device such as a diffuser can be used within the storage container that causes the bubbles of the secondary gas to be very small and numerous. In this manner, the surface area or contact area of the bubbles of the secondary gas is enhanced with the polymerized nanocomposite material 30.

Finally, the parameters of temperature and pressure within the purging storage container can be adjusted such that the desired concentration of the secondary gas and primary gas are constant and fall within a desired range. In this example, the vessel can be a separate container from the typical storage container such as a gas cylinder, or the typical storage container can be used as the purging vessel depending on the requirements of the specific application.

When released from the polymerized nanocomposite material 30, the gas flows out of the vessel, by suitable means such as a discharge port or opening 24 in FIG. 2. A flow control valve 26 may be joined in fluid communication with the interior volume of the vessel. A pipe, conduit hose, channel or other suitable device or assembly by which the fluid can be flowed out of the vessel may be connected to the vessel.

The present invention also provides a fluid storage and dispensing system. The system includes a fluid storage and dispensing vessel configured to selectively dispense a fluid therefrom. A suitable vessel is, for example, a container that can hold up to 1000 liters. A typical vessel size is about 44 liters. The vessel should be able to contain fluids at a pressure of up to about 2000 psi, preferably up to about 4000 psi. However, the vessel may also operate at around sub-atmospheric to atmospheric pressure. Preferably, the container is made of carbon steel, stainless steel, nickel or aluminum. In some cases the vessel may contain interior coatings in the form of inorganic coatings such as silicon and carbon, metallic coatings such as nickel, organic coatings such as paralyene or Teflon® coating based materials. The vessel contains a polymerized nanocomposite material 30 which reversibly takes up the fluid when contacted therewith. The fluid is releasable from the polymerized nanocomposite material 30 under dispensing conditions.

The fluids which may be stored, purified, or stabilized or any combination thereof, in the polymerized nanocomposite material 30 include, but are not limited to, alcohols, aldehydes, amines, ammonia, aromatic hydrocarbons, arsenic pentafluoride, arsine, boron trichloride, boron trifluoride, carbon dioxide, carbon disulfide, carbon monoxide, carbon sulfide, chlorine, diborane, dichlorosilane, digermane, dimethyl disulfide, dimethyl sulfide, disilane, ethane, ethers, ethylene oxide, fluorine, germane, germanium methoxide, germanium tetrafluoride, hafnium methylethylamide, hafnium t-butoxide, halogenated hydrocarbons, halogens, hexane, hydrogen, hydrogen cyanide, hydrogen halogenides, hydrogen selenide, hydrogen sulfide, ketones, metal halides, mercaptans, methane, nitric oxides, nitrogen, nitrogen trifluoride, noble gases, organometallics, oxygen, oxygenated-halogenated hydrocarbons, phosgene, phosphine, phosphorus trifluoride, n-silane, pentakisdimethylamino tantalum, propane, silicon tetrachloride, silicon tetrafluoride, stibine, styrene, sulfur dioxide, sulfur hexafluoride, sulfur tetrafluoride, tetramethyl cyclotetrasiloxane, titanium diethylamide, titanium dimethylamide, trichlorosilane, trimethyl silane, tungsten hexafluoride, water, and mixtures thereof.

In another embodiment, the fluids which may be stored, purified, or stabilized, or any combination thereof, in the polymerized nanocomposite material 30 includes a subset of the previous listed fluids and include alcohols, aldehydes, amines, ammonia, aromatic hydrocarbons, arsenic pentafluoride, arsine, boron trichloride, boron trifluoride, carbon disulfide, carbon monoxide, carbon sulfide, chlorine, diborane, dichlorosilane, digermane, dimethyl disulfide, dimethyl sulfide, disilane, ethers, ethylene oxide, fluorine, germane, germanium methoxide, germanium tetrafluoride, hafnium methylethylamide, hafnium t-butoxide, halogenated hydrocarbons, halogens, hexane, hydrogen, hydrogen cyanide, hydrogen halogenides, hydrogen selenide, hydrogen sulfide, ketones, mercaptans, nitric oxides, nitrogen, nitrogen trifluoride, organometallics, oxygenated-halogenated hydrocarbons, phosgene, phosphine, phosphorus trifluoride, n-silane, pentakisdimethylamino tantalum, silicon tetrachloride, silicon tetrafluoride, stibine, styrene, sulfur dioxide, sulfur hexafluoride, sulfur tetrafluoride, tetramethyl cyclotetrasiloxane, titanium diethylamide, titanium dimethylamide, trichlorosilane, trimethyl silane, tungsten hexafluoride, and mixtures thereof.

By way of illustration, examples of some of these classes of fluids will now be listed. However, scope of the invention is not limited to the following examples. Alcohols include ethanol, isopropanol, and methanol. Aldehydes include acetaldehyde. Amines include dimethylamine and monomethylamine. Aromatic compounds include benzene, toluene, and xylene. Ethers include dimethyl ether, and vinyl methyl ether. Halogens include chlorine, fluorine, and bromine. Halogenated hydrocarbons include dichlorodifluoromethane, tetrafluoromethane, clorodifluoromethane, trifluoromethane, difluoromethane, methyl fluoride, 1,2-dichlorotetrafluoroethane, hexafluoroethane, pentafluoroethane, halocarbon 134a tetrafluoroethane, difluoroethane, perfluoropropane, octafluorocyclobutane, chlorotrifluoroethylene, hexafluoropropylene, octafluorocyclopentane, perfluoropropane, 1,1,1-trichloroethane, 1,1,2-trichloroethane, methyl chloride, and methyl fluoride. Ketones include acetone. Mercaptans include ethyl mercaptan, methyl mercaptan, propyl mercaptan, and n,s,t-butyl mercaptan. Nitrogen oxides include nitrogen oxide, nitrogen dioxide, and nitrous oxide. Organometallics include trimethylaluminum, triethylaluminum, dimethylethylamine alane, trimethylamine alane, dimethylaluminum hydride, tritertiarybutylaluminum, tritertiarybutylaluminum trimethylindium (TMI), trimethylgallium (TMG), triethylgallium (TEG), dimethylzinc (DMZ), diethylzinc (DEZ), carbontetrabromide (CBr₄), diethyltellurium (DETe) and magnesocene (Cp₂Mg). Metal halides include transition metals along with aluminum, gallium, indium, thallium, silicon, germanium, tin, bismith in combination with one or more halogen moieties such as fluorine, chlorine, bromine, and iodine. Oxygenated-halogenated-hydrocarbons include perfluoroethylmethylether, perfluoromethylpropylether, perfluorodimethoxymethane, and hexafluoropropylene oxide. Other fluids include vinyl acetylene, acrylonitrile, and vinyl chloride.

Other fluids which may be stored, purified, or stabilized in polymerized nanocomposite material 30 include materials used for thin film deposition applications. Such materials include, but are not limited to, tetramethyl cyclotetrasiloxane (TOMCTS), titanium dimethylamide (TDMAT), titanium diethylamide (TDEAT), hafnium t-butoxide (Hf(OtBu)₄), germaniummethoxide (Ge(OMe)₄), pentakisdimethylamino tantalum (PDMAT) hafnium methylethylamide (TEMAH) and mixtures thereof.

The fluids which may be stored in the polymerized nanocomposite material 30 may be divided into categories including include stable gases, stable liquefied gases, unstable gases, and unstable liquefied gases. The term stable is relative and includes gases which do not substantially decompose over the shelf life of a storage vessel at the typical temperatures and pressures at which those skilled in the art would store the gases. Unstable refers to materials which are prone to decomposition or reaction under typical storage conditions and thus are difficult to store.

Stable gases include nitrogen, argon, helium, neon, xenon, krypton; hydrocarbons include methane, ethane, and propanes; hydrides include silane, disilane, arsine, phosphine, germane, ammonia; corrosives include hydrogen halogenides such as hydrogen chloride, hydrogen bromide, and hydrogen fluoride, as well as chlorine, dichlorosilane, trichlorosilane, carbon tetrachloride, boron trichloride, tungsten hexafluoride, and boron trifluoride; oxygenates include oxygen, carbon dioxide, nitrous oxide, and carbon monoxide; and other gases such as hydrogen, deuterium, dimethyl ether, sulfur hexafluoride, arsenic pentafluoride, and silicon tetrafluoride.

Stable liquefied gases include inerts such as nitrogen and argon; hydrocarbons such as propane; hydrides such as silane, disilane, arsine, phosphine, germane, and ammonia; fluorinates such as hexafluoroethane, perfluoropropane, and perfluorobutane; corrosives such as hydrogen chloride, hydrogen bromide, hydrogen fluoride, chlorine, dichlorosilane, trichlorosilane, carbon tetrachloride, boron trichloride, boron trifluoride, tungsten hexafluoride, and chlorine trifluoride; and oxygenates such as oxygen and nitrous oxide.

Unstable gases include digermane, borane, diborane, stibene, disilane, hydrogen selenide, nitric oxide, fluorine and organometallics including alanes, trimethyl aluminum and other similar gases. These unstable gases may also be liquefied.

In one embodiment, a fluid such as fluorine could be stored with fully fluorinated ionic liquid such as perfluorinated ammonium hexafluorophosphate.

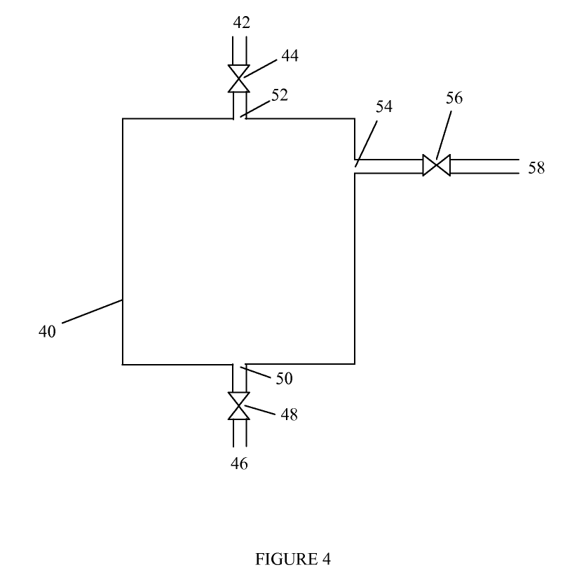

The present invention also provides a method of separating an impurity from a fluid mixture. In this instance, the fluid mixture includes a fluid and the impurity. FIG. 4 shows an embodiment of a device 40 for purifying a fluid with a polymerized nanocomposite material. A device containing the polymerized nanocomposite material is configured for contacting the polymerized nanocomposite material with the fluid mixture. A source 46 for the fluid mixture is controlled by valve 48. The fluid mixture is introduced through inlet 50 into the device 40 and contacted with the polymerized nanocomposite material. The polymerized nanocomposite material in a powdered or granular form is introduced through inlet 52 from polymerized nanocomposite material source 42 by valve 44. A portion of the impurities is retained within the polymerized nanocomposite material to produce a purified fluid. The purified fluid is released from the device through outlet 54, which is controlled by valve 56 through a discharge port or opening 58.

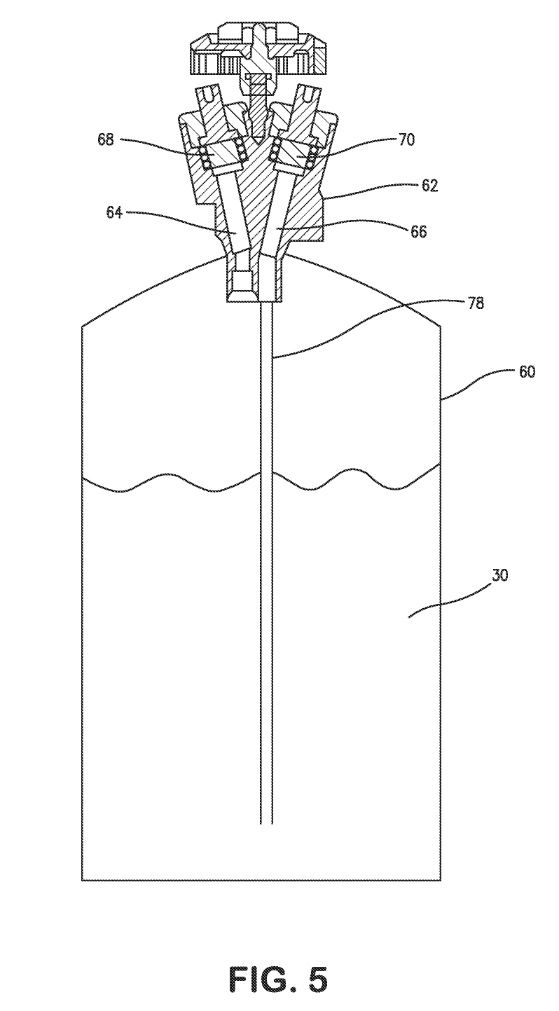

FIG. 5 shows another embodiment of a device 40 for purifying a fluid with a polymerized nanocomposite material. Contacting the fluid with the polymerized nanocomposite material comprises flowing the fluid mixture through the polymerized nanocomposite material. The vessel 60 includes a valve assembly 62, a polymerized nanocomposite material inlet 64, a fluid inlet 66, and a dip tube 78. The valve assembly 62 includes a polymerized nanocomposite material inlet valve 68 and a fluid inlet valve 70. The vessel 60 is charged with a polymerized nanocomposite material 30 through inlet 64. The vessel 60 is charged with a fluid through inlet 66 and through dip tube 78, from whence it flows through polymerized nanocomposite material 30.

It is understood that the fluid and fluid mixture may include liquids, vapors (volatilized liquids), gaseous compounds, and/or gaseous elements. Furthermore, while reference is made to “purified,” it is understood that purified may include purification to be essentially free of one or more impurities, or simply lowering the level of impurities in the fluid mixture. Impurities include any substance that may be desirable to have removed from the fluid mixture, or are undesirable within the fluid mixture. Impurities included can be variants or analogs of the fluid itself if they are undesirable. Impurities that would typically be desired to be removed include but are not limited to water, CO₂, oxygen, CO, NO, NO₂ N₂O₄, SO₂, SO₃, SO, S₂O₂, SO₄, and mixtures thereof. Additionally, impurities include but are not limited to derivatives of the fluid of interest. For example, higher boranes are considered impurities within diborane. Disilane is considered an impurity in silane. Phosphine could be considered an impurity in arsine, and HF could be considered an impurity in BF₃.

Contacting the polymerized nanocomposite material with the fluid mixture may be accomplished in any of the variety of ways. The process is selected to promote intimate mixing of the polymerized nanocomposite material and the fluid mixture and is conducted for a time sufficient to allow significant removal of targeted components. Thus, systems maximizing surface area contact between the polymerized nanocomposite material and the fluid mixture are desirable.

In an effort to maximize the surface area contact between the polymerized nanocomposite material and the fluid mixture the nanocomposite materials may be prepared as planar or curved surfaces or as free standing articles, as well as many other configurations which will become evident based on this disclosure of the present invention. Furthermore the nanocomposite materials preferably have a high surface area layer containing pores with a high effective surface area, and thus increasing the number of storage sites on the nanocomposite. The nanocomposite materials are capable of forming as nanotubes, nanofibers, nanocylinders, and arrays of nanostructured materials, of predeterminable distribution, structure, morphology, composition, and functionality.

In another aspect of the invention, a method of stabilizing an unstable fluid is provided which uses a small amount of polymerized nanocomposite material. The unstable fluid is contacted with the polymerized nanocomposite material for the purpose of stabilization only and not for uptake of the fluid by the polymerized nanocomposite material. Thus, a device or vessel is used to contact a small amount of polymerized nanocomposite material with the fluid. In this manner, a substantially less amount of polymerized nanocomposite material could be required to obtain the stabilization effect compared to an illustration wherein the unstable fluid could be taken up within the polymerized nanocomposite material. No decomposition products, or substantially less decomposition products, are produced as a result of the contact of the unstable fluid with the polymerized nanocomposite material, producing a stabilized fluid.

The present invention also provides a method for both storing and purifying a fluid mixture comprising a fluid and an impurity. A vessel contains an polymerized nanocomposite material and is configured for contacting the polymerized nanocomposite material with the fluid mixture. The fluid and the polymerized nanocomposite material may be any of the previously mentioned fluids and ionic polymerized nanocomposite materials. The fluid is contacted with the polymerized nanocomposite material for take-up of the fluid by the polymerized nanocomposite material. This may be accomplished by any of the previously described methods. A portion of the impurities is retained within the polymerized nanocomposite material to produce a purified fluid. The purified fluid can then be released from the device.

The present invention also provides a method of storing and stabilizing an unstable fluid. The unstable fluid may be any of the previously mention unstable fluids, or any other fluid that tends to decompose or react. The unstable fluid is contacted with the polymerized nanocomposite material for take-up of the unstable fluid by the polymerized nanocomposite material. The unstable fluid may be then stored within the polymerized nanocomposite material for a period of time, during which period of time the reaction or decomposition rate is at least reduced, and preferably there is substantially no decomposition of the unstable fluid. In one embodiment, the rate of decomposition is reduced by at least about 50%, more preferably at least about 75%, and most preferably at least about 90%, compared with storage of the fluid under the same temperature and pressure conditions without using a polymerized nanocomposite material. In the context of an unstable fluid, substantially no decomposition means that less than 10% of the molecules of the unstable fluid undergo a chemical change while being stored. In one embodiment, the proportion of molecules that undergo a decomposition reaction is preferably less than 1%, more preferably less than 0.1%, and most preferably less than 0.01%. Although it is most preferable for the decomposition rate to be less than 0.01%, it should be noted that in certain applications a rate of decomposition of less than 50% over the storage period of the fluid would be useful. The period of time may range from a few minutes to several years, but is preferably at least about 1 hour, more preferably at least about 24 hours, even more preferably at least about 7 days, and most preferably at least about 1 month.

The unstable fluid may be selected from categories such as dopants, dielectrics, etchants, thin film growth, cleaning, and other semiconductor processes. Examples of unstable fluids include, but are not limited to, digermane, borane, diborane, disilane, fluorine, halogenated oxyhydrocarbons, hydrogen selenide, stibene, nitric oxide, organometallics and mixtures thereof.

The present invention also provides a method of storing and purifying a fluid mixture. The storage vessel is provided with a purifying solid or liquid for contact with the fluid mixture. The purifying solid or liquid retains at least a portion of the impurity in the fluid mixture to produce a purified fluid when the fluid is released from the storage vessel. The purifying solid or liquid may be used with any of the previously mentioned fluids and polymerized nanocomposite materials.

Various purifying materials may be used with the present invention. The purification or impurity removal can be used to remove impurities from the polymerized nanocomposite material which could change the solubility of a fluid in the polymerized nanocomposite material. Alternatively, the purification material could remove only impurities present in the incoming gas or contributed from the fluid storage vessel that will be stored in the polymerized nanocomposite material. Finally, the purification material could have the ability to remove impurities from both the fluid of interest and the polymerized nanocomposite material simultaneously. The purification materials include, but are not limited to, alumina, amorphous silica-alumina, silica (SiO₂), aluminosilicate molecular sieves, titania (TiO₂), zirconia (ZrO₂), and carbon. The materials are commercially available in a variety of shapes of different sizes, including, but not limited to, beads, sheets, extrudates, powders, tablets, etc. The surface of the materials can be coated with a thin layer of a particular form of the metal (e.g., a metal oxide or a metal salt) using methods known to those skilled in the art, including, but not limited to, incipient wetness impregnation techniques, ion exchange methods, vapor deposition, spraying of reagent solutions, co-precipitation, physical mixing, etc. The metal can consist of alkali, alkaline earth or transition metals. Commercially available purification materials includes a substrate coated with a thin layer of metal oxide (known as NHX-Plus™) for removing H₂O, CO₂ and O₂, H₂S and hydride impurities, such as silane, germane and siloxanes; ultra-low emission (ULE) carbon materials (known as HCX™) designed to remove trace hydrocarbons from inert gases and hydrogen; macroreticulate polymer scavengers (known as OMA™ and OMX-Plus™) for removing oxygenated species (H₂O, O₂, CO, CO₂, NOₓ, SOₓ, etc.) and non-methane hydrocarbons; and inorganic silicate materials (known as MTX™) for removing moisture and metals. All of these are available from Matheson Tri-Gas®, Newark, Calif. Further information on these purifying materials and other purification materials is disclosed in U.S. Pat. Nos. 4,603,148; 4,604,270; 4,659,552; 4,696,953; 4,716,181; 4,867,960; 6,110,258; 6,395,070; 6,461,411; 6,425,946; 6,547,861; and 6,733,734, the contents of which are hereby incorporated by reference. Other solid purification materials typically available from Aeronex, Millipore, Mykrolis, Saes Getters, Pall Corporation, Japan Pionics and used commonly in the semiconductor gas purification applications are known in the art and are intended to be included within the scope of the present invention.

Additionally, any of the previously described storage, stabilization, and purification methods and systems may be combined to provide multiple effects. One, two or all three methods can be independently combined to obtain a process that is best suited for the application of interest. Therefore, it is conceivable that any one method or the combination of any of the methods could be used for different requirements and applications. The basic steps of these combined methods will now be set forth. It will be apparent that the information previously described for the individual methods will also be applicable for the combined methods described below. The fluids and the polymerized nanocomposite material used in the combined processes may be any of the previously mentioned fluids and polymerized nanocomposite material.

The storage method may be combined with the method of purifying using a purifying solid. In this method, a vessel containing a polymerized nanocomposite material is provided. The fluid mixture is contacted with the polymerized nanocomposite material for take-up of the fluid by the polymerized nanocomposite material. A portion of the impurity is retained by the purifying solid to produce a purified fluid.

The methods of storage, stabilizing, and purifying using a purifying solid may also be combined. A vessel containing a polymerized nanocomposite material is provided. The fluid mixture is contacted with the polymerized nanocomposite material for take-up of the fluid mixture by the polymerized nanocomposite material. A purifying solid is provided for contact with the fluid mixture. A portion of the impurity is retained by the purifying solid to produce a purified fluid. The polymerized nanocomposite material is stored for a period of time of at least about 1 hour, during which period of time there is substantially no degradation of the unstable fluid.

The methods of storage, stabilizing, and purifying using the ionic liquid may also be combined. A device containing a polymerized nanocomposite material and configured for contacting the polymerized nanocomposite material with the fluid mixture is provided. The fluid mixture is introduced into the device. The fluid mixture is contacted with the polymerized nanocomposite material. The fluid mixture may then be stored within the polymerized nanocomposite material for a period of time of at least about 1 hour, during which period of time there is substantially no degradation of the said fluid. A portion of the impurities are retained within the polymerized nanocomposite material to produce a purified fluid, and the purified fluid may then be released from the device.

The two purification methods may also be combined. A device containing a polymerized nanocomposite material and a purifying solid therein for contact with the fluid mixture is provided. The fluid mixture is introduced into the device. The fluid mixture is contacted with the polymerized nanocomposite material and with the purifying solid. A first portion of the impurity is retained within the nanocomposite material and a second portion of the impurity is retained by the purifying solid, to produce a purified fluid. The purified fluid may then be released from the device.

The storage method may be combined with both methods of purifying. A vessel containing a polymerized nanocomposite material and a purifying solid therein for contact with the fluid mixture is provided. The fluid is contacted with the polymerized nanocomposite material for take-up of the fluid by the polymerized nanocomposite material. A first portion of the impurity is retained within the nanocomposite material and a second portion of the impurity is retained by the purifying solid, to produce a purified fluid. The purified fluid may then be released from the device.

The storage and stabilization methods may be combined with both methods of purifying. A vessel containing a polymerized nanocomposite material and a purifying solid therein for contact with the fluid mixture is provided. The fluid mixture is introduced into the device. The fluid is contacted with the polymerized nanocomposite material for take-up of the fluid by the polymerized nanocomposite material. The fluid mixture is stored within the polymerized nanocomposite material for a period of time of at least about 1 hour, during which period of time there is substantially no degradation of the unstable fluid. A first portion of the impurity is retained within the polymerized nanocomposite material and a second portion of the impurity is retained by the purifying solid, to produce a purified unstable fluid. The purified fluid may then be released from the device.

The stabilization methods may be combined with both methods of purifying. A vessel containing a polymerized nanocomposite material and a purifying solid therein for contact with the fluid mixture is provided. The unstable fluid mixture is introduced into the device. The unstable fluid is contacted with the polymerized nanocomposite material primarily for the purposes of stabilization and purification only, and not for the purposes of uptake of the fluid by the polymerized nanocomposite material. Thus, a device or vessel is used to contact a small amount of polymerized nanocomposite material with the fluid. In this manner, a substantially less amount of polymerized nanocomposite material could be required to obtain the stabilization effect and the purification effect compared to the previous illustrations wherein the unstable fluid could be taken up by the polymerized nanocomposite material. No decomposition products, or substantially less decomposition products, are produced as a result of the contact of the unstable fluid with the polymerized nanocomposite material, producing a stabilized fluid. The fluid mixture is stored within the polymerized nanocomposite material for a period of time of at least about 1 hour, during which period of time there is substantially no degradation of the unstable fluid. A portion of the impurity is retained within the polymerized nanocomposite material or purifying solid to produce a purified fluid. The purified fluid may then be released from the device.

### EXAMPLES

The invention is further illustrated by the following non-limited examples. All scientific and technical terms have the meanings as understood by one with ordinary skill in the art. The specific examples which follow illustrate the methods in which the methodology of the present invention may be preformed and are not to be construed as limiting the invention in sphere or scope. The methods may be adapted to variation in order to produce compositions embraced by this invention but not specifically disclosed. Further, variations of the methods to produce the same compositions in somewhat different fashion will be evident to one skilled in the art.

### Example 1

### Storage of a Gas Using Nanocomposite Material in which the Solvent is an Ionic Liquid—BF₃ stored in poly[1,1′-[1,2-ethanediylbis(oxy-2,1-ethanediyl)]-2,2′-undecyl-3,3′-(undecyl-11-acryloyloxy)-bisimidazolium di-tetrafluoroborate]. 1-ethyl-3-methylimidazolium tetrafluoroborate

A stainless steel canister is charged with a known quantity of the nanocomposite material poly(1,1′-[1,2-ethanediylbis(oxy-2,1-ethanediyl)]-2,2′-undecyl-3,3′-(undecyl-11-acryloyloxy)-bisimidazolium di-tetrafluoroborate). 1-ethyl-3-methylimidazolium tetrafluoroborate. The charged canister is thermally controlled by a PID temperature controller or variac with a heating element and a thermocouple. The canister is placed on a gravimetric load cell or weight scale and a pressure gauge is connected to the canister to measure head pressure. This canister is connected to a manifold with vacuum capability and to a gas source. The canister is also connected to an analyzer (such as FT-IR, GC, APIMS, etc.).

A vacuum bake procedure is conducted on the canister, charged with poly(1,1′-[1,2-ethanediylbis(oxy-2,1-ethanediyl)]-2,2′-undecyl-3,3′-(undecyl-11-acryloyloxy)-bisimidazolium di-tetrafluoroborate). 1-ethyl-3-methylimidazolium tetrafluoroborate and the manifold up to the source gas cylinder, by pulling a vacuum while heating. This removes any trace moisture and other volatile impurities from the poly(1,1′-[1,2-ethanediylbis(oxy-2,1-ethanediyl)]-2,2′-undecyl-3,3′-(undecyl-11-acryloyloxy)-bisimidazolium di-tetrafluoroborate). 1-ethyl-3-methylimidazolium tetrafluoroborate material, and the gas distribution components. Under vacuum, the charged canister is allowed to cool to the desired operating temperature. The mass of the vacuum baked canister containing the poly(1,1′-[1,2-ethanediylbis(oxy-2,1-ethanediyl)]-2,2′-undecyl-3,3′-(undecyl-11-acryloyloxy)-bisimidazolium di-tetrafluoroborate). 1-ethyl-3-methylimidazolium tetrafluoroborate is recorded.

The source gas, BF₃ or a gas mixture containing BF₃ is then introduced into the canister, at 5 psig, until the uptake of BF₃ is at the desired level. The uptake can be determined gravimetrically, by pressure, or by analytical methods. For example, BF₃ will continue to be introduced until the pressure has reached a predetermined desired pressure, such as 670 Torr. At this time, the source gas flow is stopped. The mass of the BF₃ filled canister is recorded. The increase in mass of the charged canister now filled with BF₃ is the amount of BF₃ stored.

The BF₃ filled canister is stored for a period of time. It is then heated, a pressure differential is applied, or it is purged with an inert gas, in order to deliver the stored BF₃.

### Example 2

### Storage of a Gas Using Nanocomposite Material in which the Solvent is a Molecular Solvent—BF₃ Stored in poly[1,1′-[1,2-ethanediylbis(oxy-2,1-ethanediyl)]-2,2′-undecyl-3,3′-(undecyl-11-acryloyloxy)-bisimidazolium dibromide]. H₂O

A stainless steel canister is charged with a known quantity of the nanocomposite material poly[1,1′-[1,2-ethanediylbis(oxy-2,1-ethanediyl)]-2,2′-undecyl-3,3′-(undecyl-11-acryloyloxy)-bisimidazolium dibromide]. H₂O. The charged canister is thermally controlled by a PID temperature controller or variac with a heating element and a thermocouple. The canister is placed on a gravimetric load cell or weight scale and a pressure gauge is connected to the canister to measure head pressure. This canister is connected to a manifold with vacuum capability and to a gas source. The canister is also connected to an analyzer (such as FT-IR, GC, APIMS, etc.).

A vacuum bake procedure is conducted on the canister, charged with poly[1,1′-[1,2-ethanediylbis(oxy-2,1-ethanediyl)]-2,2′-undecyl-3,3′-(undecyl-11-acryloyloxy)-bisimidazolium dibromide]H₂O material, and the manifold up to the source gas cylinder, by pulling a vacuum while heating. This removes any trace moisture and other volatile impurities from the poly[1,1′-[1,2-ethanediylbis(oxy-2,1-ethanediyl)]-2,2′-undecyl-3,3′-(undecyl-11-acryloyloxy)-bisimidazolium dibromide]. H₂O material and the gas distribution components. Under vacuum, the charged canister is allowed to cool to the desired operating temperature. The mass of the vacuum baked canister containing the poly[1,1′-[1,2-ethanediylbis(oxy-2,1-ethanediyl)]-2,2′-undecyl-3,3′-(undecyl-11-acryloyloxy)-bisimidazolium dibromide]. H₂O material is recorded.

The source gas, BF₃ or a gas mixture containing BF₃ is then introduced into the canister, at 5 psig, until the uptake of BF₃ is at the desired level. The uptake can be determined gravimetrically, by pressure, or by analytical methods. For example, BF₃ will continue to be introduced until the pressure has reached a predetermined desired pressure. At this time, the source gas flow is stopped. The mass of the BF₃ filled canister is recorded. The increase in mass of the charged canister now filled with BF₃ is the amount of BF₃ stored.

The BF₃ filled canister is stored for a period of time. It is then heated, a pressure differential is applied, or it is purged with an inert gas, in order to deliver the stored BF₃.

### Example 3

### Storage of a Gas Using Nanocomposite Material in which the Solvent is a mixture of Ionic Liquid and Molecular Solvent—BF₃ stored in poly[1,1′-[1,2-ethanediylbis(oxy-2,1-ethanediyl)]-2,2′-undecyl-3,3′-(undecyl-11-acryloyloxy)-bisimidazolium di-bromide]1-ethyl-3-methylimidazolium bromide. H₂O

A stainless steel canister is charged with a known quantity of the nanocomposite material poly[1,1′-[1,2-ethanediylbis(oxy-2,1-ethanediyl)]-2,2′-undecyl-3,3′-(undecyl-11-acryloyloxy)-bisimidazolium di-bromide]. 1-ethyl-3-methylimidazolium bromide. H₂O. The charged canister is thermally controlled by a PID temperature controller or variac with a heating element and a thermocouple. The canister is placed on a gravimetric load cell or weight scale and a pressure gauge is connected to the canister to measure head pressure. This canister is connected to a manifold with vacuum capability and to a gas source. The canister is also connected to an analyzer (such as FT-IR, GC, APIMS, etc.).

A vacuum bake procedure is conducted on the canister, charged with poly[1,1′-[1,2-ethanediylbis(oxy-2,1-ethanediyl)]-2,2′-undecyl-3,3′-(undecyl-11-acryloyloxy)-bisimidazolium di-bromide]. 1-ethyl-3-methylimidazolium bromide H₂O and the manifold up to the source gas cylinder, by pulling a vacuum while heating. This removes any trace moisture and other volatile impurities from the poly[1,1′-[1,2-ethanediylbis(oxy-2,1-ethanediyl)]-2,2′-undecyl-3,3′-(undecyl-11-acryloyloxy)-bisimidazolium di-bromide]. 1-ethyl-3-methylimidazolium bromide H₂O material, and the gas distribution components. Under vacuum, the charged canister is allowed to cool to the desired operating temperature. The mass of the vacuum baked canister containing the poly[1,1′-[1,2-ethanediylbis(oxy-2,1-ethanediyl)]-2,2′-undecyl-3,3′-(undecyl-11-acryloyloxy)-bisimidazolium di-bromide]. 1-ethyl-3-methylimidazolium bromide H₂O is recorded.

The source gas, BF₃ or a gas mixture containing BF₃ is then introduced into the canister, at 5 psig, until the uptake of BF₃ is at the desired level. The uptake can be determined gravimetrically, by pressure, or by analytical methods. For example, BF₃ will continue to be introduced until the pressure has reached a predetermined desired pressure. At this time, the source gas flow is stopped. The mass of the BF₃ filled canister is recorded. The increase in mass of the charged canister now filled with BF₃ is the amount of BF₃ stored.

The BF₃ filled canister is stored for a period of time. It is then heated, a pressure differential is applied, or it is purged with an inert gas, in order to deliver the stored BF₃.

The foregoing description is considered as illustrative only of the principles of the invention.

The words “comprise,” “comprising,” “include,” “including,” and “includes” when used in this specification and in the following claims are intended to specify the presence of one or more stated features, integers, components, or steps, but they do not preclude the presence or addition of one or more other features, integers, components, steps, or groups thereof. Furthermore, since a number of modifications and changes will readily will readily occur to those skilled in the art, it is not desired to limit the invention to the exact construction and process shown described above. Accordingly, all suitable modifications and equivalents may be resorted to falling within the scope of the invention as defined by the claims which follow.

All references cited herein are hereby incorporated by reference in their entireties, whether previously specifically incorporated or not. As used herein, the terms “a”, “an,” and “any” are each intended to include both the singular and plural forms.

1. A method of storing and dispensing a fluid, comprising:
a) providing a vessel configured for selective dispensing of the fluid therefrom;
b) providing a nanocomposite material within the vessel wherein said nanocomposite material comprises a surfactant and an integral solvent that is essential to the formation of said nanocomposite material;
c) contacting the fluid with said nanocomposite material for take-up of the fluid by the solvent mixture;
d) releasing the fluid from said nanocomposite material; and
e) dispensing the fluid from the vessel
wherein the surfactant is a cationic imidazolium surfactant.

2. The method of claim 1, wherein the surfactant comprises an anion selected from the group consisting of Br⁻, BF₄⁻, Cl⁻, I⁻, CF₃SO₃⁻, Tf₂N⁻, PF₆⁻, DCA⁻, MeSO₃⁻, and TsO⁻.

3. The method of claim 1, wherein the surfactant comprises an anion selected from the group consisting of

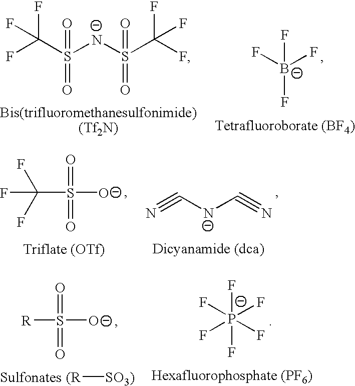

4. The method of claim 1, wherein the nanocomposite material is polymerized.

5. The method of claim 1 wherein said solvent is selected from the group consisting of water, ionic liquids, molecular solvents and mixtures thereof.

6. The method of claim 5, wherein said molecular solvent is selected from the group consisting of aliphatics, aromatics, acetone, acetonitrile, aldehydes, amines, amides, aniline, alcohols, benzene, benzoyl chloride, butanol, carbon disulfide, carbon tetrachloride, chlorobenzene, chloroform, cyclohexane, cyclohexanol, dichloroethane, diethylether, dimethoxyethane, dimethylformamide, esters, ethers, ethanol, ethylacetate, heptane, hexane, ketones, methanol, methylacetate, methylene chloride, nitriles, nitrobenzene, pentane, propanol, pyridine, tetrahydrofuran, thiols, toluene.

7. The method of claim 1 wherein said solvent is an ionic liquid.

8. The method of claim 1 wherein the solvent is a mixture of ionic liquids.

9. The method of claim 1 wherein said surfactant material has the formula:
HₙXₙL₍ₙ₋₁₎Yₙ
where n is greater than or equal to 2; H is a hydrophilic head group comprising a five membered aromatic ring containing two nitrogens; X is an anion, L is a spacer or linking group which connects two rings, and Y is a hydrophobic tail group attached to each ring and having at least 10 carbon atoms which optionally comprise a polymerizable group P.

10. The method of claim 9 wherein n is 2 and said spacer L is attached to a first nitrogen atom in each of the two linked rings, through a covalent or a noncovalent bond.

11. The method of claim 10 wherein said hydrophobic tail group Y is attached to the second (other) nitrogen atom in each ring, wherein the combination of the hydrophilic head group H, the linker L, and the hydrophobic tail Y form an imidazolium cation.

12. The method of claim 11 wherein a hydrophobic tail is also attached to one or more carbon atoms of the ring.

13. The method of claim 9, wherein the anion, X, is selected from the group consisting of Br⁻, BF₄⁻, Cl⁻, I⁻, CF₃SO₃⁻, Tf₂N⁻, PF₆⁻, DCA⁻, MeSO₃⁻, and TsO⁻.

14. The method of claim 9 wherein said spacer L is an alkyl group, an ether group, an amide, an ester, an anhydride, a phenyl group, a perfluoroalkyl, a perfluoroether, or a siloxane.

15. The method of claim 14 wherein said spacer L is an alkyl group having from 1 to about 12 carbons, or an ether group having from 1 to about 6 ethers.

16. The method of claim 15 wherein said spacer L is an ether group having from 1 to 3 ethers.

17. The method of claim 9 wherein Y is a linear alkyl chain.

18. The method of claim 17, wherein Y comprises a polymerizable group.

19. The method of claim 18 wherein said polymerizable groups are selected from the group consisting of acrylate, methacrylate, diene, vinyl, (halovinyl), styrenes, vinylether, hydroxyl groups, epoxy or other oxiranes (halooxirane), dienoyls, diacetylenes, styrenes, terminal olefins, isocyanides, acrylamides, and cinamoyl groups.

20. The method of claim 9 wherein n=2 and the surfactant composition has the general formula:

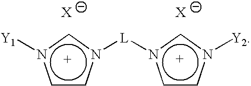

21. The method of claim 9 wherein n=2 and the surfactant composition has the general formula: 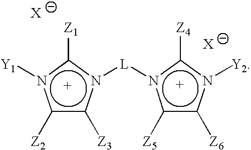
wherein Z₁ through Z₆ are individually selected from the group consisting of hydrogen and hydrophobic tail groups having at least 10 carbon atoms which optionally comprise a polymerizable group P.

22. The method of claim 1 wherein said nanocomposite material comprises a surfactant having the formula: 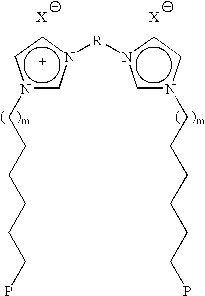
Wherein P is a polymerizable group, R is —(CH₂)ₜ— or —(OCH₂)ₜ—; X is Br⁻ or BF₄⁻; t is 1-12, u is 1-6 and m is 0-6; in combination with an integral solvent that is essential to the formation of said nanocomposite material.

23. The method of claim 1, wherein the surfactant has the general formula: 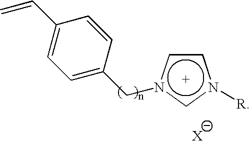
wherein n≧1, X is an anion and R is selected from the group consisting of an alkyl chain with a formula range of CH₃ to C₁₈H₃₇, an oligo (ethylene glycol) unit with a formula range of C₃H₇O to C₁₁H₂₃O₅, perfluoroalkyl, siloxane, nitrile, ester, aromatic and cyclic units.

24. The method of claim 1, wherein the surfactant has the general formula: 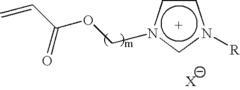
wherein m≧2, X is an anion, and R is selected from the group consisting of an alkyl chain with a formula range of CH₃ to C₁₈H₃₇, an oligo (ethylene glycol) unit with a formula range of C₃H₇O to C₁₁H₂₃O₅, perfluoroalkyl, siloxane, nitrile, ester, aromatic and cyclic units.

25. The method of claim 1, wherein the surfactant has the general formula: 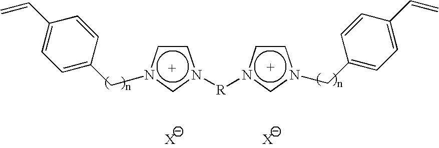
wherein n≧1, X is an anion and R is selected from the group consisting of an alkyl chain with a formula range of CH₂ to C₁₈H₃₆, an oligo (ethylene glycol) chain with a formula range of C₄H₈O to C₁₄H₂₈O₆, perfluoroalkyl, siloxane, nitrile, ester, aromatic and cyclic units.

26. The method of claim 1, wherein the surfactant has the general formula: 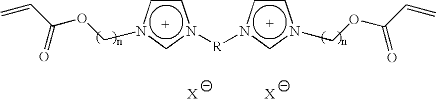
wherein n≧2, X is an anion and R selected from the group consisting of an alkyl chain with a formula range of CH₂ to C₁₈H₃₆, an oligo (ethylene glycol) chain with a formula range of C₄H₈O to C₁₄H₂₈O₆, perfluoroalkyl, siloxane, nitrile, ester, aromatic and cyclic units.

27. A storage device for a fluid, comprising:
a vessel configured for selective dispensing of the fluid therefrom; and a nanocomposite material positioned within the vessel wherein, said nanocomposite material comprises a surfactant and an integral solvent that is essential to the formation of said nanocomposite material, wherein the surfactant is a cationic imidazolium surfactant.

28. The storage device of claim 27, wherein the surfactant comprises an anion selected from the group consisting of Br⁻, BF₄⁻, Cl⁻, I⁻, CF₃SO₃⁻, Tf₂N⁻, PF₆⁻, DCA⁻, MeSO₃⁻, and TsO⁻.

29. The storage device of claim 27, wherein the surfactant comprises an anion selected from the group consisting of

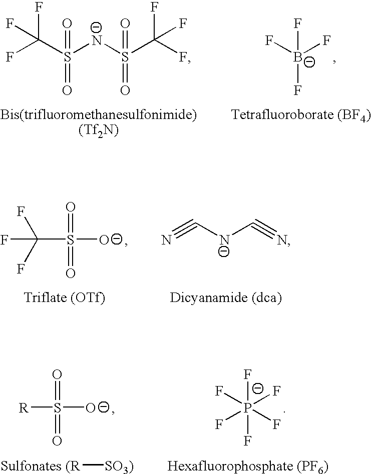

30. The storage device of claim 27, wherein the nanocomposite material is polymerized.

31. The storage device of claim 27 wherein said solvent is selected from the group consisting of water, ionic liquids, molecular solvents and mixtures thereof.

32. The method of claim 31, wherein said molecular solvent is selected from the group consisting of aliphatics, aromatics, acetone, acetonitrile, aldehydes, amines, amides, aniline, alcohols, benzene, benzoyl chloride, butanol, carbon disulfide, carbon tetrachloride, chlorobenzene, chloroform, cyclohexane, cyclohexanol, dichloroethane, diethylether, dimethoxyethane, dimethylformamide, esters, ethers, ethanol, ethylacetate, heptane, hexane, ketones, methanol, methylacetate, methylene chloride, nitriles, nitrobenzene, pentane, propanol, pyridine, tetrahydrofuran, thiols, toluene

33. The storage device of claim 27 wherein said solvent is an ionic liquid.

34. The storage device of claim 27 wherein the solvent is a mixture of ionic liquids.

35. The storage device of claim 27 wherein said surfactant material has the formula:
HₙXₙL₍ₙ₋ₗ₎Yₙ
where n is greater than or equal to 2; H is a hydrophilic head group comprising a five membered aromatic ring containing two nitrogens; X is an anion, L is a spacer or linking group which connects two rings, and Y is a hydrophobic tail group attached to each ring and having at least 10 carbon atoms which optionally comprise a polymerizable group P.

36. The storage device of claim 35 wherein n is 2 and said spacer L is attached to a first nitrogen atom in each of the two linked rings, through a covalent or a noncovalent bond.

37. The storage device of claim 36 wherein said hydrophobic tail group Y is attached to the second (other) nitrogen atom in each ring, wherein the combination of the hydrophilic head group H, the linker L, and the hydrophobic tail Y form an imidazolium cation.

38. The storage device of claim 37 wherein a hydrophobic tail is also attached to one or more carbon atoms of the ring.

39. The storage device of claim 35, wherein the anion, X, is selected from the group consisting of Br⁻, BF₄⁻, Cl⁻, I⁻, CF₃SO₃⁻, Tf₂N⁻, PF₆⁻, DCA⁻, MeSO₃⁻, and TsO⁻.

40. The storage device of claim 35 wherein said spacer L is an alkyl group, an ether group, an amide, an ester, an anhydiride, a phenyl group, a perfluoroalkyl, a perfluoroether, or a siloxane.

41. The storage device of claim 40 wherein said spacer L is an alkyl group having from 1 to about 12 carbons, or an ether group having from 1 to about 6 ethers.

42. The storage device of claim 41 wherein said spacer L is an ether group having from 1 to 3 ethers.

43. The storage device of claim 35 wherein Y is a linear alkyl chain.

44. The storage device of claim 43, wherein Y comprises a polymerizable group.

45. The storage device of claim 44 wherein said polymerizable groups are selected from the group consisting of acrylate, methacrylate, diene, vinyl, (halovinyl), styrenes, vinylether, hydroxyl groups, epoxy or other oxiranes (halooxirane), dienoyls, diacetylenes, styrenes, terminal olefins, isocyanides, acrylamides, and cinamoyl groups.

46. The storage device of claim 35 wherein n=2 and said surfactant has the general formula:

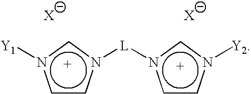

47. The storage device of claim 35 wherein n=2 and said surfactant has the general formula: 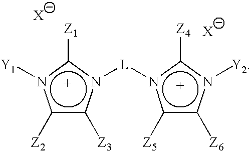
wherein Z₁ through Z₆ are individually selected from the group consisting of hydrogen and hydrophobic tail groups having at least 10 carbon atoms which optionally comprise a polymerizable group P.

48. The storage device of claim 27 wherein said nanocomposite material has the formula: 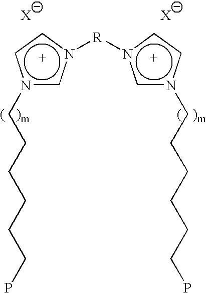
Wherein P is a polymerizable group, R is —(CH₂)ₜ— or —(OCH₂)ₜ—; X is Br⁻ or BF₄⁻; t is 1-12, u is 1-6 and m is 0-6; in combination with an integral solvent that is essential to the formation of said nanocomposite material.

49. The storage device of claim 27, wherein the surfactant has the general formula: 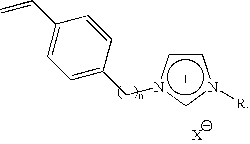
wherein n≧1, X is an anion and R is selected from the group consisting of an alkyl chain with a formula range of CH₃ to C₁₈H₃₇, an oligo (ethylene glycol) unit with a formula range of C₃H₇O to C₁₁H₂₃O₅, perfluoroalkyl, siloxane, nitrile, ester, aromatic and cyclic units.

50. The storage device of claim 27, wherein the surfactant has the general formula: 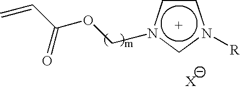
wherein m≧2, X is an anion, and R is selected from the group consisting of an alkyl chain with a formula range of CH₃ to C₁₈H₃₇, an oligo (ethylene glycol) unit with a formula range of C₃H₇O to C₁₁H₂₃O₅, perfluoroalkyl, siloxane, nitrile, ester, aromatic and cyclic units.

51. The storage device of claim 27, wherein the surfactant has the general formula: 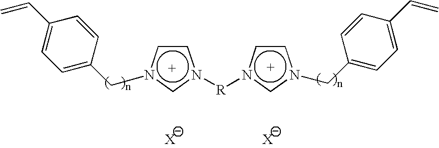
wherein n≧1, X is an anion and R is selected from the group consisting of an alkyl chain with a formula range of CH₂ to C₁₈H₃₆, an oligo (ethylene glycol) chain with a formula range of C₄H₈O to C₁₄H₂₈O₆, perfluoroalkyl, siloxane, nitrile, ester, aromatic and cyclic units.

52. The storage device of claim 27, wherein the surfactant has the general formula: 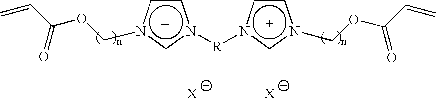
Wherein n≧2, X is an anion and R selected from the group consisting of an alkyl chain with a formula range of CH₂ to C₁₈H₃₆, an oligo (ethylene glycol) chain with a formula range of C₄H₈O to C₁₄H₂₈O₆, perfluoroalkyl, siloxane, nitrile, ester, aromatic and cyclic units.

53. A compound of formula: 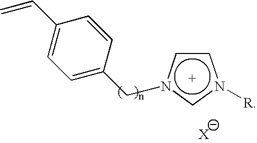
wherein n≧1, X is an anion and R is selected from the group consisting of: an alkyl chain with a formula range of CH₃—C₁₈H₃₇, an oligo (ethylene glycol) unit with a formula range of C₃H₇O—C₁₁H₂₃O₅, perfluoroalkyl, siloxane, nitrile, ester, aromatic and cyclic units.

54. The compound of claim 53 wherein X is selected from the group consisting of:

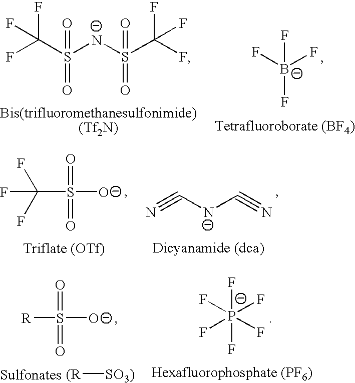

55. A compound of formula: 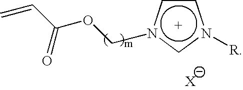
wherein m≧2, X is an anion and R is selected from the group consisting of: an alkyl chain with a formula range of CH₃—C₁₈H₃₇, an oligo (ethylene glycol) unit with a formula range of C₃H₇O—C₁₁H₂₃O₅, perfluoroalkyl, siloxane, nitrile, ester, aromatic and cyclic units.

56. The compound of claim 55 wherein X is selected from the group consisting of:

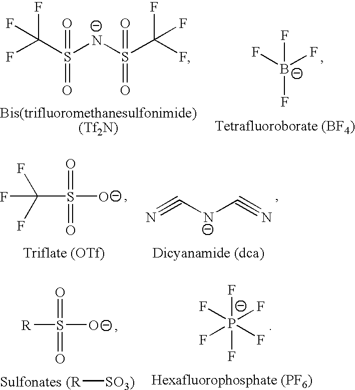

57. A compound of formula: 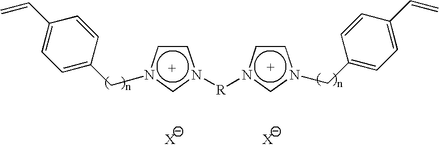
wherein n≧1, X is an anion and R is selected from the group consisting of an alkyl chain with a formula range of CH₂C₁₈H₃₆, an oligo (ethylene glycol) chain with a formula range of C₄H₈O—C₁₄H₂₈O₆, perfluoroalkyl, siloxane, nitrile, ester, aromatic and cyclic units.

58. The compound of claim 57 wherein X is selected from the group consisting of:

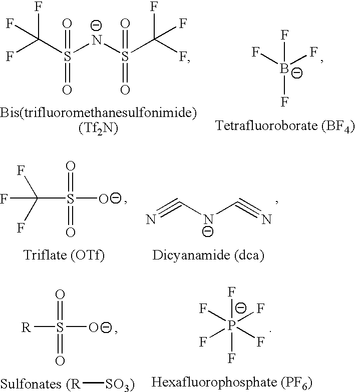

59. A compound of formula: 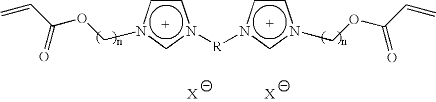
wherein n≧2, X is an anion and R is selected from the group consisting of an alkyl chain with a formula range of CH₂C₁₈H₃₆, an oligo (ethylene glycol) chain with a formula range of C₄H₈O—C₁₄H₂₈O₆, perfluoroalkyl, siloxane, nitrile, ester, aromatic and cyclic units.

60. The compound of claim 59 wherein X is selected from the group consisting of: [chemical structure]
